(19) United States
(12) Patent Application Publication
(10) Pub. No.: US 2024/0058182 A1
(43) Pub. Date: Feb. 22, 2024

(54) METHOD OF MAKING ABSORBENT STRUCTURES WITH CHANNELS

(71) Applicant: The Procter & Gamble Company, Cincinnati, OH (US)

(72) Inventors: Carsten Heinrich KREUZER, Hofheim (DE); Rodrigo ROSATI, Frankfurt am Main (DE); Blanca ARIZTI, Schmitten (DE); Hans Adolf JACKELS, Mechernich (DE); Ernesto Gabriel BIANCHI, Oberursel (DE); Donald Carroll ROE, West Chester, OH (US)

(21) Appl. No.: 18/499,508

(22) Filed: Nov. 1, 2023

Related U.S. Application Data
(63) Continuation of application No. 16/541,286, filed on Aug. 15, 2019.
Application No. 16/541,286 is a continuation of application No. 15/921,693, filed on Mar. 15, 2018, now Pat. No. 10,893,987.
Application No. 15/921,693 is a continuation of application No. 14/517,954, filed on Oct. 20, 2014, now Pat. No. 10,517,777.
Application No. 14/517,954 is a continuation of application No. 13/491,644, filed on Jun. 8, 2012, now Pat. No. 10,149,788.
(60) Provisional application No. 61/495,406, filed on Jun. 10, 2011.

(51) Int. Cl. A61F 13/534; A61F 13/533; A61F 13/536

(52) U.S. Cl. CPC A61F 13/534; A61F 13/533; A61F 13/536; A61F 2013/530343

(57) ABSTRACT

A method for making an absorbent structure for use in a disposable absorbent article is provided. The method involves the steps of providing a first moving surface having an absorbent layer-forming reservoir, providing a supply of absorbent material, and depositing a portion of the supply of absorbent material into the absorbent layer-forming reservoir. The method further involves associating the portion of the supply of absorbent material with a first substrate layer and/or a second substrate layer to define an absorbent structure having a channel therein corresponding to the one or more raised strips, and bonding the first substrate layer to the second substrate layer through the channel. The channel is substantially free of the absorbent material.

### CROSS-REFERENCE TO RELATED APPLICATIONS

This application is a continuation of, and claims priority under 35 U.S.C. § 120 to, U.S. patent application Ser. No. 16/541,286, filed on Aug. 15, 2019, which is a continuation of, and claims priority under 35 U.S.C. § 120, to U.S. patent application Ser. No. 15/921,693, filed on Mar. 15, 2018, which is a continuation of, and claims priority under 35 U.S.C. § 120, to U.S. patent application Ser. No. 14/517,954, filed on Oct. 20, 2014, which is a continuation of, and claims priority under 35 U.S.C. § 120, to U.S. patent application Ser. No. 13/491,644, filed on Jun. 8, 2012, which claims the benefit, under 35 USC 119(e), of U.S. Provisional Patent Application No. 61/495,406, filed on Jun. 10, 2011; each of which are herein incorporated by reference in their entirety.

### FIELD

The present disclosure relates to a method of making disposable diapers providing an improved fit in dry and wet states (i.e., when loaded with bodily fluids) and providing improved liquid transportation.

### BACKGROUND

Disposable absorbent articles for receiving and retaining bodily discharges such as urine or feces are generally known in the art. Examples of these include disposable diapers, training pants and adult incontinence articles. Typically, disposable diapers comprise a liquid pervious topsheet that faces the wearer's body, a liquid impervious backsheet that faces the wearer's clothing and an absorbent core interposed between the liquid pervious topsheet and the backsheet.

Since their introduction into the market place, disposable diapers have continued to improve regarding comfort, fit and functionalities.

An important component of disposable absorbent articles is the absorbent core structure. The absorbent core structure typically includes absorbent polymer material, such as hydrogel-forming polymer material, also referred to as absorbent gelling material, AGM, or super-absorbent polymer, SAP. This absorbent polymer material ensures that large amounts of bodily fluids, e.g. urine, can be absorbed by the absorbent article during its use and be locked away, thus providing low rewet and good skin dryness.

Traditionally, the absorbent polymer material is incorporated into the absorbent core structure with cellulose or cellulosic fibres. However, over the past years, significant effort has been spent to make thinner absorbent core structures which can still acquire and store large quantities of discharged body fluids, in particular urine. Hereto, it has been proposed to reduce or eliminate these cellulose fibres from the absorbent core structures. To maintain the mechanical stability of the absorbent core structures, small quantities of thermoplastic adhesive material, such as fibrous thermoplastic adhesive material, may be added to stabilize the absorbent polymer material. Resultantly, absorbent structures having the required permeability/porosity, reduced gel-blocking, and that form stable structures in use or transport are provided.

However, it was found that some absorbent core structures with reduced cellulose fibre content, whilst being very thin when not loaded with bodily fluids, may have an increased stiffness when partially loaded or fully loaded, especially in those regions which comprise most of the absorbent capacity of the absorbent article, such as the front region and crotch region of the diaper. Increased stiffness is not desirable since it reduces the absorbent article's ability to conform to the body of the wearer once worn. Furthermore, it was also found that some absorbent core structures which comprise absorbent polymer particles of high absorption capacity swell significantly upon loading with body exudates. As a result, the volume of the absorbent article may increase significantly during use, especially in these regions which comprise most of the absorbent capacity of the absorbent article, such as the front region and crotch region of the diaper. Such an increase in volume may render the disposable article uncomfortable for the wearer. It was also found that some of these absorbent core structures with reduced cellulose fibres content may have a reduced void volume which impacts negatively the liquid acquisition speed.

Therefore there is still a need for disposable diapers having good liquid handling properties and having an increased flexibility during the whole use of the article and which deliver, in particular, a better fit in the wet state.

### SUMMARY

The present disclosure generally relates to a method for making an absorbent structure for use in a disposable absorbent article. The method comprising the step of providing a first moving surface comprising an absorbent layer-forming reservoir having one or more substantially longitudinally extending raised strips, wherein the one or more substantially longitudinally extending raised strips have an average width of between 5% and 30% of an average width of the absorbent layer-forming reservoir, and wherein the first moving surface is partially air permeable. The method further comprises the steps of providing a supply of absorbent material comprising superabsorbent polymer particulate material, depositing a portion of the supply of absorbent material into the absorbent layer-forming reservoir, associating the portion of the supply of absorbent material with a first substrate layer and/or a second substrate layer to define an absorbent structure comprising a channel therein corresponding to the one or more raised strips, and bonding the first substrate layer to the second substrate layer through the channel. The channel may be substantially free of the absorbent material.

The present disclosure further relates to a disposable diaper having a transverse and longitudinal dimension. The disposable diaper comprises a backsheet, a topsheet and an absorbent core disposed therebetween. The absorbent core comprises at least one absorbent structure comprising a substrate layer and an absorbent layer. The absorbent layer comprises absorbent polymer particles, and optionally cellulose fibers, supported by and immobilized on the substrate layer. The absorbent layer has a transverse and longitudinal dimension and a thickness, a pair of opposing longitudinal edges extending in the longitudinal dimension, a pair of opposing transversal edges extending in its transverse dimension and a front, crotch and back regions arranged sequentially in the longitudinal dimension. The central longitudinal axis of the absorbent layer delimits two longitudinal portions. The absorbent layer may comprise one or more main channels substantially free of said absorbent polymer particles extending through its thickness and at least present in the crotch region; and one or more secondary channels substantially free of the absorbent material extending through the thickness of the absorbent layer. The secondary channels may be shorter than the main channels. In other embodiments, at least two main channels are separated by a first minimum transverse spacing and at least two secondary channels are separated by a second minimum transverse spacing, wherein the first minimum transverse spacing is different than the second minimum transverse spacing.

In certain embodiments, the absorbent layer does not comprise channels substantially free of absorbent polymer particles extending up to its longitudinal and transverse edges.

Further, each channel may comprise a width of at least 3 mm or of at least 4% of the transverse dimension of the absorbent layer, and/or extend over at least 15% of the longitudinal dimension of the absorbent layer. The channels may be at least present in the crotch region or part thereof. In some nonlimiting examples, the absorbent layer may not comprise channels substantially free of absorbent polymer particles extending in its transverse dimension in the crotch region.

### BRIEF DESCRIPTION OF THE DRAWINGS

FIG. 1 is a plan view of a disposable diaper in accordance with one non-limiting embodiment.

FIG. 2 is a perspective view of an absorbent structure comprising an absorbent layer with two longitudinal main channels in accordance with one non-limiting embodiment.

FIG. 3A is a perspective view of an absorbent layer comprising four longitudinal main channels in accordance with one non-limiting embodiment.

FIG. 3B is a top view of an absorbent structure comprising an absorbent layer with four longitudinal main channels in accordance with one non-limiting embodiment.

FIG. 3C is a perspective view of the absorbent structure of FIG. 3B in accordance with one non-limiting embodiment.

FIGS. 4A to 4E are schematic representations of channels in accordance with various non-limiting embodiments.

FIG. 5 is a perspective view of an absorbent layer comprising two longitudinal main channels at least present in the crotch region and two longitudinal secondary channels in the front region in accordance with one non-limiting embodiment.

FIG. 6 is a perspective view of an absorbent layer comprising two longitudinal main channels at least present in the crotch region, two longitudinal secondary channels in the front region and two longitudinal secondary channels in back front region in accordance with one non-limiting embodiment.

FIGS. 7 to 13 are schematic cross-sections of an absorbent core taken in the transverse dimension in accordance with various non-limiting embodiments.

FIG. 14 is a schematic representation of a process for making an absorbent structure comprising an absorbent layer with channels in accordance with one non-limiting embodiment.

### DETAILED DESCRIPTION

### Definitions

As used herein “diapers” refers to devices which are intended to be placed against the skin of a wearer to absorb and contain the various exudates discharged from the body. Diapers are generally worn by infants and incontinent persons about the lower torso so as to encircle the waist and legs of the wearer. Examples of diapers include infant or adult diapers and pant-like diapers such as training pants.

“Training pant”, as used herein, refers to disposable garments having a waist opening and leg openings designed for infant or adult wearers. A pant may be placed in position on the wearer by inserting the wearer's legs into the leg openings and sliding the pant into position about a wearer's lower torso. A pant may be preformed by any suitable technique including, but not limited to, joining together portions of the article using refastenable and/or non-refastenable bonds (e.g., seam, weld, adhesive, cohesive bond, fastener, etc.). A pant may be preformed anywhere along the circumference of the article (e.g., side fastened, front waist fastened).

“Disposable” is used herein to describe articles that are generally not intended to be laundered or otherwise restored or reused (i.e., they are intended to be discarded after a single use and, may be recycled, composted or otherwise disposed of in an environmentally compatible manner).

As used herein “absorbent structure” refers to a three dimensional structure useful to absorb and contain liquids, such as urine. The absorbent structure may be the absorbent core of an absorbent article or may be part of the absorbent core of an absorbent article, i.e. an absorbent component of the absorbent core, as will be further described herein.

As used herein “absorbent core” refers to a component of an absorbent article typically disposed between a topsheet and backsheet of an absorbent article. The absorbent core of an absorbent article may comprise one or more absorbent structures and optionally further layers, such as for instance a cover layer.

“Absorbent polymer particles” as used herein refers to substantially water-insoluble polymer particles that can absorb at least 10 times their weight of a 0.9% saline solution in de-mineralized water as measured using the Centrifuge Retention Capacity test (Edana 441.2-01).

“Nonwoven material” as used herein refers to a manufactured web of directionally or randomly orientated fibers, excluding paper and products which are woven, knitted, tufted, stitch-bonded incorporating binding yarns or filaments, or felted by wet-milling, whether or not additionally needled. Nonwoven materials and processes for making them are known in the art. Generally, processes for making nonwoven materials comprise laying fibers onto a forming surface, which may comprise spunlaying, meltblowing, carding, airlaying, wetlaying, coform and combinations thereof. The fibers may be of natural or man-made origin and may be staple fibers or continuous filaments or be formed in situ.

The term “basis weight” as used herein refers to the mass of a material per unit area, i.e. the mass of absorbent polymer particles per unit area, e.g. gram per square meter (gsm).

“Hot melt adhesive” as used herein refers to adhesives in alignment with the description given in “Adhesion and Adhesives Technology: An Introduction” by Alphonsus V. Pocius (Hanser publishers Munich, 1997). Therein a hot melt is defined as an adhesive applied from the melt and gaining strength upon solidification.

In the following description of the present disclosure, the surface of the disposable diaper, or of an element thereof, which faces in use in the direction of the wearer, is called the “wearer-facing surface”. Conversely, the surface facing in use in the direction of the garment is called the “garment-facing surface”. The disposable diaper, as well as any element thereof, such as, for example the absorbent structure, has therefore a wearer-facing surface and a garment-facing surface.

Unless specified otherwise, the longitudinal dimension or length of an absorbent layer as used herein is to be understood as the average length.

Unless specified otherwise, the transverse dimension or width of an absorbent layer as used herein is to be understood as the average width.

### Disposable Diapers

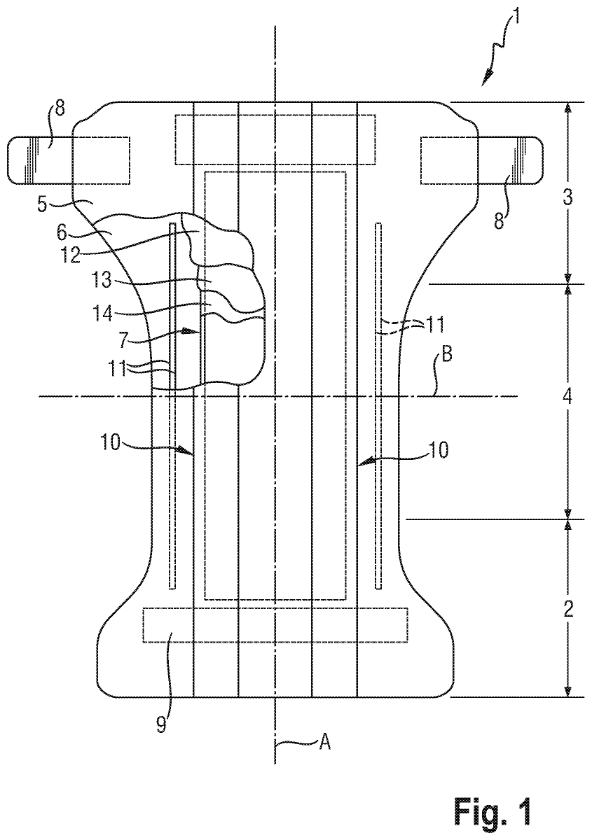

The disposable diaper 1, such as illustrated in FIG. 1, has a longitudinal dimension (along a longitudinal axis A) and a transverse dimension (along a transverse axis B) perpendicular thereto.

One end portion of the diaper is configured as a front waist region 2 (which is the front one third of the article, having one third of the length of the article). The opposite end portion is configured as a back waist region 3 (which is the back one third of the article, having one third of the length of the article). An intermediate portion of the diaper is configured as a crotch region 4 (which is the centre one third of the article). The crotch region extends longitudinally between the front and back waist regions. The crotch region is that portion of the diaper which, when the diaper is worn, is generally positioned between the wearer's legs.

The diaper typically comprises a topsheet 5, a backsheet 6 and an absorbent core 7 disposed therebetween.

The topsheet may be liquid pervious. The topsheet may be at least partially hydrophilic. So-called apertured topsheets may also be used. Topsheets with one or more (large) openings may also be used. The topsheet may also include a skin care composition, e.g., a lotion. The topsheet may be fully or partially elasticized or may be foreshortened to provide a void space between the topsheet and the absorbent core. Example structures including elasticized or foreshortened topsheets are described in more detail in U.S. Pat. No. 5,037,416 entitled “Disposable Absorbent Article Having Elastically Extensible Topsheet” issued to Allen et al. on Aug. 6, 1991; and U.S. Pat. No. 5,269,775 entitled “Trisection Topsheets for Disposable Absorbent Articles and Disposable Absorbent Articles Having Such Trisection Topsheets” issued to Freeland et al. on Dec. 14, 1993.

The backsheet may be vapor pervious but liquid impervious. The backsheet may be used to at least inhibit the fluids absorbed and contained in the absorbent core from wetting materials that contact the absorbent article such as underpants, pants, pyjamas, undergarments, and shirts or jackets, thereby acting as a barrier to fluid transport. In certain embodiments, the backsheet may be substantially impervious to liquids (e.g., urine) and comprise a laminate of a nonwoven and a thin plastic film such as a thermoplastic film having a thickness of about 0.012 mm (0.5 mil) to about 0.051 mm (2.0 mils). Suitable backsheet films include those manufactured by Tredegar Industries Inc. of Terre Haute, Ind. and sold under the trade names X15306, X10962, and X10964. Other suitable backsheet materials may include breathable materials that permit vapors to escape from the diaper while still at least inhibiting liquid exudates from passing through the backsheet. Example breathable materials may include materials such as woven webs, nonwoven webs, composite materials such as film-coated nonwoven webs, and microporous films such as manufactured by Mitsui Toatsu Co., of Japan under the designation ESPOIR NO and by EXXON Chemical Co., of Bay City, Tex., under the designation EXXAIRE. Suitable breathable composite materials comprising polymer blends are available from Clopay Corporation, Cincinnati, Ohio under the name HYTREL blend P18-3097. Such breathable composite materials are described in greater detail in PCT Application No. WO 95/16746, published on Jun. 22, 1995 in the name of E. I. DuPont. Other breathable backsheets including nonwoven webs and apertured formed films are described in U.S. Pat. No. 5,571,096 issued to Dobrin et al. on Nov. 5, 1996.

The absorbent core 7 is disposed between the topsheet and the backsheet of the absorbent article. The absorbent core may comprise one or more absorbent structures as disclosed herein.

The diaper may further comprise a front and back waist band and/or a fastening system, typically joined to the waistband, as known in the art. Desired fastening systems comprise fastening tabs 8 and landing zones 9, wherein the fastening tabs are attached or joined to the back waist region of the diaper and the landing zones are part of the front waist region of the diaper. The diaper may also have leg cuffs 10 and/or barrier cuffs, such as elasticized barrier cuffs 11. Suitable cuffs are described, for example, in U.S. Pat. Nos. 3,860,003; 4,808,178; 4,695,278 and 4,795,454.

As illustrated in FIG. 1, the absorbent core may comprise an acquisition system comprising an upper acquisition layer 12 and a lower acquisition layer 13 and optionally a core cover 14. Processes for assembling the diaper include conventional techniques generally known in the art for constructing and configuring disposable absorbent articles. For example, the backsheet and/or the topsheet can be joined to the absorbent core or to each other by a uniform continuous layer of adhesive, a patterned layer of adhesive, or an array of separate lines, spirals, or spots of adhesive. Adhesives which have been found to be satisfactory are manufactured by H. B. Fuller Company of St. Paul, Minnesota under the designation HL-1258 or H-2031. While the topsheet, the backsheet, and the absorbent core may be assembled in a variety of well-known configurations, desired diaper configurations are described generally in U.S. Pat. No. 5,554,145 entitled “Absorbent Article With Multiple Zone Structural Elastic-Like Film Web Extensible Waist Feature” issued to Roe et al. on Sep. 10, 1996; U.S. Pat. No. 5,569,234 entitled “Disposable Pull-On Pant” issued to Buell et al. on Oct. 29, 1996; and U.S. Pat. No. 6,004,306 entitled “Absorbent Article With Multi-Directional Extensible Side Panels” issued to Robles et al. on Dec. 21, 1999.

As indicated above, the absorbent core may comprise one or more absorbent structures that absorb and contain liquids, such as urine. The absorbent structure may be the absorbent core of an absorbent article or may be part of the absorbent core of an absorbent article.

### Absorbent Structure

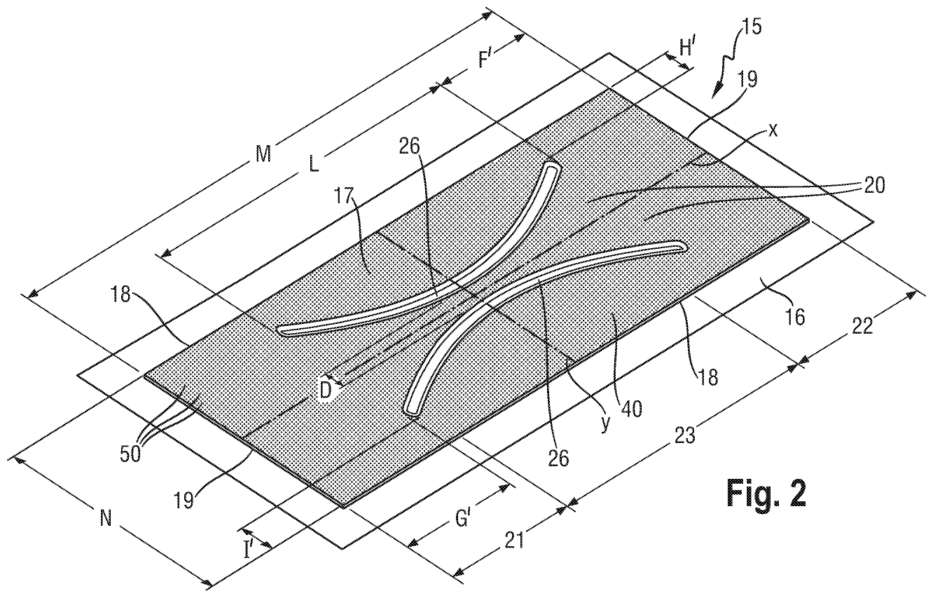

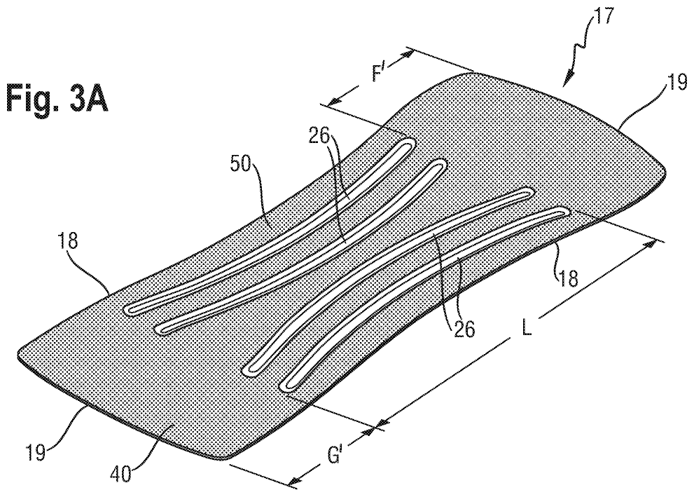

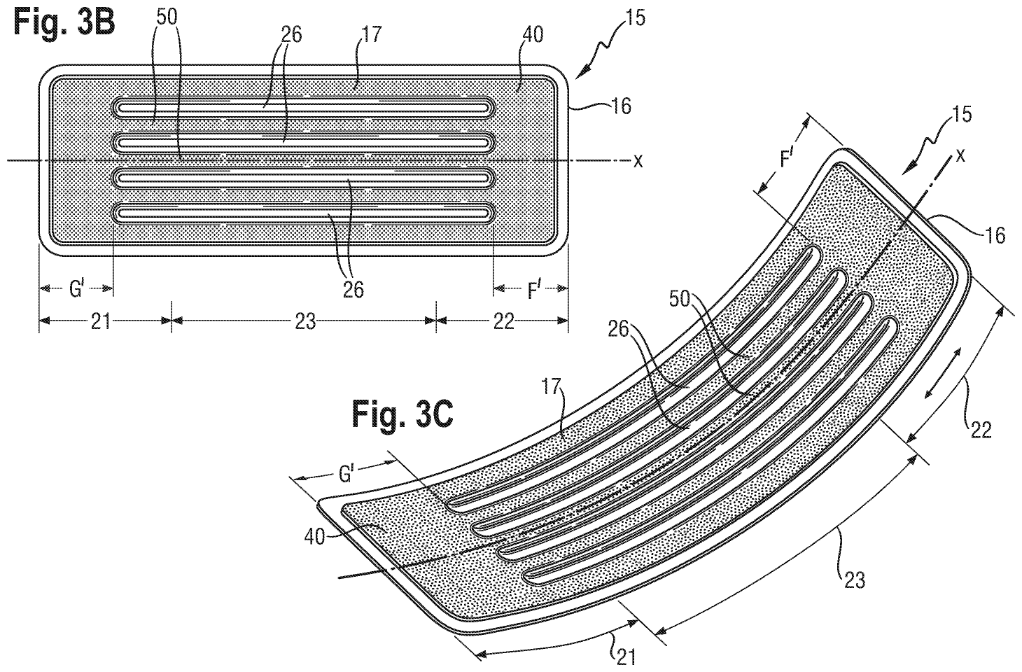

The absorbent structure 15 is a three-dimensional structure comprising a substrate layer 16 and an absorbent layer 17 comprising absorbent polymer particles, and optionally cellulose, supported by, and immobilized on, said substrate layer 16. Examples of absorbent structures 15 are illustrated in FIGS. 2, 3B and 3C.

The substrate layer has a longitudinal dimension extending in the longitudinal dimension of the diaper and a transverse dimension extending in the transverse dimension of the diaper

The absorbent layer has a longitudinal dimension M extending in the longitudinal dimension of the diaper (i.e. the absorbent layer has a length M) and a transverse dimension N extending in the transverse dimension of the diaper (i.e. the absorbent layer has a width N). The absorbent layer possesses a central longitudinal axis x, a central transverse axis y perpendicular to said central longitudinal axis x, a pair of opposing longitudinal edges 18 extending in the longitudinal dimension of the disposable diaper and a pair of opposing transverse edges 19 extending in the transverse dimension of the disposable diaper. The longitudinal edges or transverse edges of the absorbent layer may be parallel respectively to the central longitudinal axis or central transverse axis (as shown in FIG. 2) or they may follow the general direction of these axes while not being strictly parallel, e.g. they may be curvilinear as for instance to provide for a narrower transverse dimension in the crotch region (as shown in FIGS. 3A, 5 and 6).

The central longitudinal axis x of the absorbent layer 17 delimits two regions of the absorbent layer referred herein as longitudinal regions 20 (the plane perpendicular to the central longitudinal axis divides the absorbent layer 17 in two longitudinal regions 20 disposed on either side of said plane).

One end portion of the absorbent layer is configured as a front region 21 (which is the region oriented toward the front waist region of the disposable diaper) which makes up 25% of the longitudinal dimension M of the absorbent layer 17. The opposite end region is configured as a back region 22 (which is the region oriented toward the back waist region of the diaper) which makes up 25% of the longitudinal dimension M of the absorbent layer 17. An intermediate portion of the absorbent layer 17 is configured as a crotch region 23 which makes up 50% of the longitudinal dimension M of the absorbent layer. The front, crotch and back regions are arranged sequentially in the longitudinal dimension of the absorbent layer.

The substrate layer of the absorbent structure may be any material capable to support the absorbent polymer particles. Typically, it is a web or sheet material, such as foam, film woven and/or nonwoven material. “Nonwoven material” as used herein refers to a manufactured web of directionally or randomly orientated fibers, bonded by friction, and/or cohesion and/or adhesion, excluding paper and products which are woven, knitted, tufted, stitch-bonded incorporating binding yarns or filaments, or felted by wet-milling, whether or not additionally needled. Nonwoven materials and processes for making them are generally known in the art. Generally, processes for making nonwoven materials comprise two steps: fiber laying onto a forming surface and fiber bonding. The fiber laying step may comprise spunlaying, meltblowing, carding, airlaying, wetlaying, coform and combinations thereof. The fiber bonding step may comprise hydroentanglement, cold calendering, hot calendering, through air thermal bonding, chemical bonding, needle punching, and combinations thereof. The nonwoven material may be a laminate. The laminate may comprise spunbond layer(s) (S), and/or meltblown layer(s) (M), and/or carded layer(s) (C). Suitable laminates include, but are not limited to, SS, SSS, SMS or SMMS. The nonwoven material may have a basis weight from about 5 to 100 g/m², or from about 10 to 40 g/m², or from about 10 to 30 g/m². Woven or nonwoven materials may comprise natural fibers or synthetic fibers or combinations thereof. Examples of natural fibers may include cellulosic natural fibers, such as fibers from hardwood sources, softwood sources, or other non-wood plants. The natural fibers may comprise cellulose, starch and combinations thereof. The synthetic fibers can be any material, such as, but not limited to, those selected from the group consisting of polyolefins (polypropylene and polypropylene copolymers, polyethylene and polyethylene copolymers), polyesters (e.g., polyethylene terephthalate), polyethers, polyamides, polyesteramides, polyvinylalcohols, polyhydroxyalkanoates, polysaccharides, and combinations thereof. Further, the synthetic fibers can be a single component (i.e. a single synthetic material or a mixture that makes up the entire fiber), bi-component (i.e. the fiber is divided into regions, the regions including two or more different synthetic materials or mixtures thereof and may include co-extruded fibers and core and sheath fibers) and combinations thereof. Bi-component fibers can be used as a component fiber of the nonwoven material, and/or they may be present to act as a binder for the other fibers present in the nonwoven material. Any or all of the fibers may be treated before, during, or after manufacture to change any desired properties of the fibers.

The substrate layer 16 and the absorbent layer 17 may be coextensive or the substrate layer 16 may be slightly longer and wider than the absorbent layer 17 (as shown in FIGS. 2, 3B and 3C.

The absorbent layer 17 comprises absorbent polymer particles 50, and optionally cellulose. Absorbent polymer particles will be described in further details herein below. The absorbent polymer particles may be used alone or in combination with other materials. In some embodiments, the absorbent layer comprises absorbent polymer particles combined with cellulose. “Cellulose” as used herein refers to comminuted wood pulp in the form of fibers, typically also referred in the art as “air-felt”. In some embodiments, the absorbent layer comprises more than 70%, or more than 80%, or more than 90%, or more than 95% or even 100% by weight of absorbent polymer particles. In some other embodiments, the absorbent layer comprises absorbent polymer particles and less than 5% by weight of cellulose, more typically less than 2% by weight of cellulose and most typically the absorbent layer is cellulose free. In embodiments wherein the absorbent layer is cellulose free, the absorbent layer comprises only absorbent polymer particles. The resulting absorbent structures have a reduced thickness in the dry state compared to conventional absorbent structure comprising cellulosic fibers. The reduced thickness helps to improve the fit and comfort of the absorbent article for the wearer.

The absorbent layer 17 comprises at least two channels 26 substantially free of absorbent polymer particles extending through the thickness of the absorbent layer in the longitudinal dimension of the absorbent layer. By extending in the longitudinal dimension of the absorbent layer, it is meant that the channels extend essentially in the longitudinal dimension, i.e. they extend more in the longitudinal dimension than in the transverse dimension, e.g. at least twice as much in the longitudinal dimension than in the transverse dimension. These two channels are referred herein as “longitudinal main channels”.

In addition to these two longitudinal main channels 26, the absorbent layer 17 may comprise further channels 26′, referred herein as “secondary channels”.

“Channels” as used herein refer to discrete portions of the absorbent layer extending through the thickness of the absorbent layer which are substantially free of absorbent polymer particles, i.e. no absorbent polymer particles are intentionally present in such a channel (longitudinal main channel or secondary channel) of an absorbent structure. However, it should be understood that, accidentally, a small, negligible amount of absorbent polymer particles may be present in the channel, which may not contribute to the overall functionality (e.g. absorbency of the absorbent structure). Typically, the channels possess two transverse edges (in the shortest dimension) and two longitudinal edges (in the longest dimension) running between the transverse edges. The transverse edges of the channels may be straight (i.e. perpendicular to the longitudinal side edges), angled or curved. The channels have an average width w of at least 3 mm (the average of a channel is defined as the average distance between the longitudinal side edges) or of at least 4% of the width of the absorbent layer. In some embodiments, the longitudinal channels may have complex shapes: the channels may not end with a straight, angled or curved traverse edge but may have one or more ramifications at one or the two of their end edges (referred herein as ramified channels). The ramifications also possess longitudinal edges and one transverse edge. The channels, as well as their ramifications, have an average width w of at least 3 mm or of at least 4% of the width of the absorbent layer.

The channels may be permanent. By permanent, it is meant that the integrity of the channels is at least partially maintained both in dry state and wet state, i.e. the channels are resistant to frictions caused by movements of the diaper's wearer and resist wetting by bodily fluids, such as urine. Permanent channels are obtained by immobilizing the absorbent polymer particles on the substrate layer, such as by applying a thermoplastic adhesive material over the absorbent layer. Alternatively, the channels may be made permanent by folding the substrate layer into the channels or allowing the substrate layer to enter the channels so as to immobilize the absorbent polymer particles as will be described in further details herein below.

The absorbent cores (7) of the present disclosure may comprise in particular permanent channels formed by bonding of the first substrate layer (16) and second substrate layer (16′) through the channels. Typically, glue may be used to bond both substrate layers through the channel, but it is possible to bond via other known means, for example ultrasonic bonding, or heat bonding. The supporting layers can be continuously bonded or intermittently bonded along the channels.

The Wet Channel Integrity Test described below can be used to test if channels are permanent following wet saturation and to what extent.

### Wet Channel Integrity Test

This test is designed to check the integrity of a channel following wet saturation. The test can be performed directly on an absorbent structure or on an absorbent core containing the absorbent structure.
1. The length (in millimeters) of the channel is measured in the dry state (if the channel is not straight, the curvilinear length through the middle of the channel is measured).
2. The absorbent structure or core is then immersed in 5 liters of synthetic urine “Saline”, with a concentration of 9.00 g NaCl per 1000 ml solution prepared by dissolving the appropriate amount of sodium chloride in distilled water. The temperature of the solution must be 20+/−5° C.
3. After 1 minute in the saline, the absorbent structure or core is removed and held vertically by one end for 5 seconds to drain, then extended flat on a horizontal surface with the garment-facing side down, if this side is recognizable. If the absorbent structure or core comprises stretch elements, the absorbent structure or core is pulled taut in both X and Y dimensions so that no contraction is observed. The extremes/edges of the absorbent structure or core are fixed to the horizontal surface, so that no contraction can happen.
4. The absorbent structure or core is covered with a suitably weighted rigid plate, with dimensions as follows: length equal to the extended length of the absorbent structure or core, and width equal to the maximum absorbent structure or core width in the cross direction.
5. A pressure of 18.0 kPa is applied for 30 seconds over the area of the rigid plate above mentioned. Pressure is calculated on the basis of overall area encompassed by the rigid plate. Pressure is achieved by placing additional weights in the geometric center of the rigid plate, such that the combined weight of the rigid plate and the additional weights result in a pressure of 18.0 kPa over the total area of the rigid plate.
6. After 30 seconds, the additional weights and the rigid plate are removed.
7. Immediately afterwards, the cumulative length of the portions of the channel which remained intact is measured (in millimeters; if the channel is not straight, the curvilinear length through the middle of the channel is measured). If no portions of the channel remained intact then the channel is not permanent.
8. The percentage of integrity of the permanent channel is calculated by dividing the cumulative length of the portions of the channel which remained intact by the length of the channel in the dry state, and then multiplying the quotient by 100.

Advantageously, a permanent channel according to the present disclosure has a percentage of integrity of at least 20%, or 30%, or 40%, or 50%, or 60, or 70%, or 80%, or 90% following this test.

When the absorbent structure comprises absorbent polymer particles and cellulose, it may be desired that said channels, i.e. longitudinal main channels and/or secondary channels, are also free of such cellulose.

In the following, when applicable, the description applies to each channel taken independently. For example, by “two longitudinal channels may extend over a distance L which is at least 15%”, it is meant that each of the two longitudinal channels may extend over a distance L which is at least 15% . . . ”, i.e. the channels may be the same or different.

### Longitudinal Main Channels

The two longitudinal main channels 26 may be distributed in the absorbent layer 17 such that each longitudinal portion 20 of the absorbent layer comprises one longitudinal main channel 26.

As shown in FIG. 2, the two longitudinal main channels are at least present in the crotch region of the absorbent layer. By “at least present in the crotch region”, it is meant that the channels may be only present in the crotch region or they may extend from the crotch region up to the front region and/or up to the back region, i.e. they may extend beyond the crotch region. In some embodiments, the two longitudinal main channels may extend across at least 15%, or at least 20% or at least 30% and up to 50%, or up to 70% or up to 90% of the longitudinal dimension of the absorbent layer (i.e. they may extend over a distance L which is at least 15% and up to 50%, or up to 70% or up to 90% of the length M of the absorbent layer). In some embodiments, the two longitudinal main channels may be present only in the crotch region. When present only in the crotch region, the longitudinal main channels may extend over the whole longitudinal dimension of the crotch region, i.e. 50% of the longitudinal dimension M of the absorbent layer, or they may extend in only part of the crotch region, i.e. from at least 15%, or at least 20% or at least 30% to 40%, or to 45% or to less than 50% of the longitudinal dimension of the absorbent layer. In some embodiments, the two longitudinal main channels 26 may be present in the crotch region, or part thereof, and part of the front region and/or part of the back region (such as shown in FIG. 2). In some embodiments, the longitudinal main channels may be present in the front and crotch regions, i.e. the channels extend through the crotch region (or part thereof) and part of the front region. In these embodiments, the longitudinal main channels may extend up to 70% of the longitudinal dimension of the absorbent layer, typically from 15%, or from 30%, or from 35% or from 40% to 70% of the longitudinal dimension of the absorbent layer (i.e. they may extend over a distance L which is up to 70% of the length M of the absorbent layer). In some embodiments, the longitudinal main channels may be present in the back and crotch regions, i.e. the channels extend through the crotch region (or part thereof) and part of the back region. In these embodiments, the longitudinal main channels may extend up to 70% of the longitudinal dimension of the absorbent layer, typically from 15%, or from 30%, or from 35% or from 40% to 70% of the longitudinal dimension of the absorbent layer (i.e. they may extend over a distance L which is up to 70% of the length M of the absorbent layer). In some embodiments, the longitudinal main channels may be present in the front, crotch and back regions. In these embodiments, the longitudinal main channels may extend up to 90% of the longitudinal dimension M of the absorbent layer, typically from 55% or from 60% to 70%, or to 80% of the longitudinal dimension of the absorbent layer (i.e. they may extend over a distance L which is up to 90% of the length M of the absorbent layer).

The longitudinal main channels 26 may be minor images of one another with respect to the central longitudinal axis x of the absorbent layer 17, i.e. the longitudinal main channel in one longitudinal region 20 may be minor image of the longitudinal main channel in the other longitudinal region of the absorbent layer 17.

The longitudinal main channels 26 may not extend up to the transverse edges 19 of the absorbent layer 17, i.e. from one transverse edge to the other. Typically, the absorbent layer comprises, along each transverse edge and immediately adjacent to said edge, a strip free of channels which extends in the transverse dimension of the absorbent layer from one longitudinal edge to the other. Said strips have respectively a width F′ or G′ which is at least 5% of the longitudinal dimension of the absorbent layer (i.e. a width which is at least 5% of the length of the absorbent layer). In other words, the smallest distance F′ or G′ between the edge of a channel and the transverse edge of the absorbent layer is at least 5% of the longitudinal dimension M of the absorbent layer. In some embodiments, the width F′ or G′ is at least from 5% to 15%, or to 10% of the longitudinal dimension of the absorbent layer.

Furthermore, in order to reduce the risk of fluid leakages and run-off, the longitudinal main channels may not extend up to the longitudinal edges 18 of the absorbent layer 17. Typically, the absorbent layer comprises, along each longitudinal edge and immediately adjacent to said edge, a strip free of channel which extends in the longitudinal dimension of the absorbent layer from one transverse edge to the other. Said strips have respectively a width I′ or H′ which is at least 5%, or at least 10%, or at least 12% to 25% of the transverse dimension N of the absorbent layer in a given region (i.e. a width I′ or H′ which is at least 5% of the width N of the absorbent layer). In other words, the minimum distance I′ or H′ between the edge of a channel and the longitudinal edge of the absorbent layer is at least from 5% to 25% of the transverse dimension of the absorbent layer. For example, the distance I′ or H′ in the crotch region may correspond to at least 5%, or to at least 10% or at least 12% of the transverse dimension N of the absorbent layer in said crotch region. In some embodiments, the distance I′ and/or H′ is of 10 mm, or 15 mm or 20 mm.

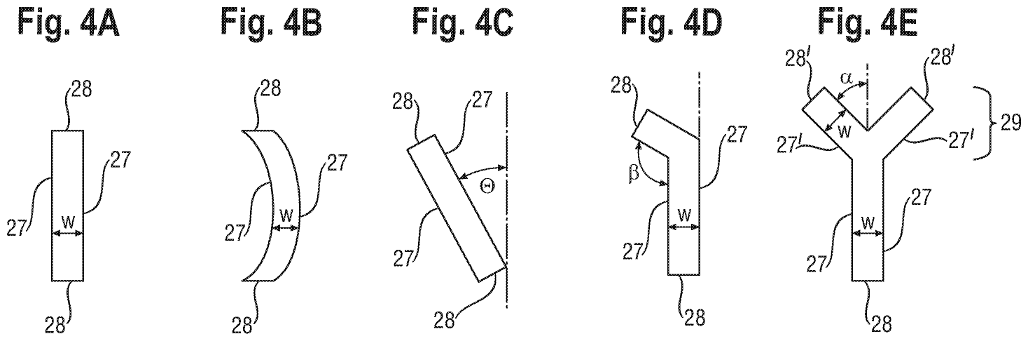

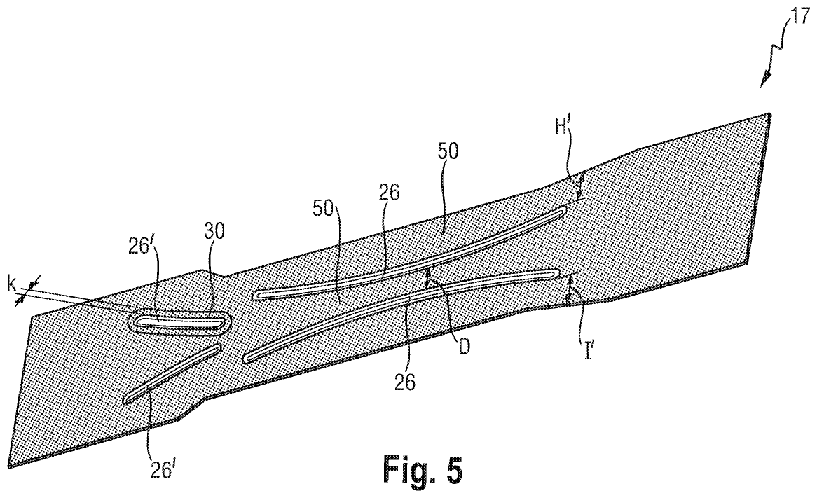

The longitudinal main channels may be straight channels running parallel to the longitudinal axis of the absorbent layer (as shown schematically in FIG. 4A). Straight channels act as folding lines in the absorbent structure which contribute to provide a desirable bucket shape to the diaper once worn. The diaper once worn conforms to the inside of the wearer's thigh. As a result, a U-like shape is achieved which minimizes leakage and increases comfort. These channels also improve fluid transportation within the absorbent structure and therefore contribute to fast insult acquisition.

Alternatively, the longitudinal main channels may be curved, as shown in FIG. 4B. Curved channels act as folding lines in the absorbent structure which assist the absorbent structure in following the morphology of the diaper's wearer, i.e. the channels constrain the product to take a U-like shape when the diaper is worn and compressed by the wearer's tights. Thus, the channels provide a comfortable and superior fit in addition to permitting improved liquid transportation.

The longitudinal main channels 26 may be oblique channels, as shown in FIG. 4C, i.e. straight channels oriented under an angle θ of up to 30 degrees, or up to 20 degrees, or up to 10 degrees with respect to the longitudinal central axis of the absorbent structure.

In some other alternatives, the longitudinal main channels may be angled channels, as shown in FIG. 4D. Angled channels are channels made of two or more portions connected under an angle σ to one another. Typically, angled channels are made of two portions connected under an angle σ of at least 150 degrees, or at least 160 degrees or at least 170 degrees.

In some embodiments, the longitudinal main channels may be so-called “ramified” channels, i.e. channels having at least one extremity which may not end with a straight, angled or curved transverse edge 28 but with a ramification 29. The ramification 29 may form an angle α of up to 30 degrees, or up to 20 degrees or up to 10 degrees with respect to the longitudinal central axis of the channel.

The longitudinal main channels 26 may have an average width w of from 3 mm to 15 mm, or from 4 mm to 14 mm or from 5 mm to 12 mm (the average width of a channel is the average distance between its longitudinal side edges 27). The average width of the longitudinal main channels may be at least 4% of the width of the absorbent layer, or at least 7% and up to 15%, or 20% or 25%. In some embodiments, the longitudinal main channels may have an average width w of from 3 mm to 18 mm, or from 5 mm to 15 mm or from 6 to 10 mm. The ramifications 29, as well, have an average width w of at least 3 mm or of at least 4% of the width of the absorbent layer, or of at least 7% and up to 15%, or up to 20% or up to 25% (average distance between the longitudinal edges 27′ of the ramifications 29).

The longitudinal main channels 26 may be separated in the crotch region by a distance D (shown in FIG. 2) of at least 5%, or at least 10%, or at least 20%, or at least 25% of the transverse dimension (width) of the absorbent layer in said crotch region. It was found that when these two longitudinal main channels are separated by a distance of at least 5% of the transverse dimension of the absorbent layer in the crotch region, the disposable diaper takes a desirable bucket shape which improves the fit of the diaper. In some embodiments, the longitudinal main channels may be separated in the crotch region by a distance of at least 10 mm, or at least 15 mm, or at least 20 mm, or at least 30 mm. In some embodiments, the distance separating the longitudinal main channels in the crotch region is from 20 to 30 mm.

The inventors have found that when an absorbent structure is provided with at least two channels as described above, i.e. two longitudinal main channels, the flexibility of the absorbent structure is increased, especially for absorbent structure comprising exclusively absorbent polymer particles in the absorbent layer. The two channels create bending lines which drive the bending of the diaper to conform to the wearer's anatomy and therefore improve the fit of the diaper.

Furthermore, the inventors observed that absorbent structure having at least two channels as described above exhibit better fluid transportation versus absorbent structure of the same type not comprising channels. Indeed, it was observed that the channels provide for fast insults acquisition which reduces risk of leakages. The channels avoid saturation of the absorbent layer in the region of fluid discharge which increases the risk of leakages.

### Secondary Channels

The absorbent layer may comprise further channels 26′ to further increase the fluid transportation and/or fit of the absorbent article, referred herein as secondary channels. The above description of the longitudinal main channels may equally apply to any of said secondary channels 26′. However, in some embodiments, the secondary channels may be shorter than the main longitudinal channels.

The secondary channels may extend in the longitudinal dimension of the absorbent layer (longitudinal secondary channels) and/or in the transverse dimension of the absorbent layer (transverse secondary channels), provided they do not extend up to the longitudinal edges and/or transverse edges of the absorbent layer. Thus, the absorbent layer is free of channels, i.e. longitudinal main channels and secondary channels, extending up to its longitudinal edges and its transverse edges.

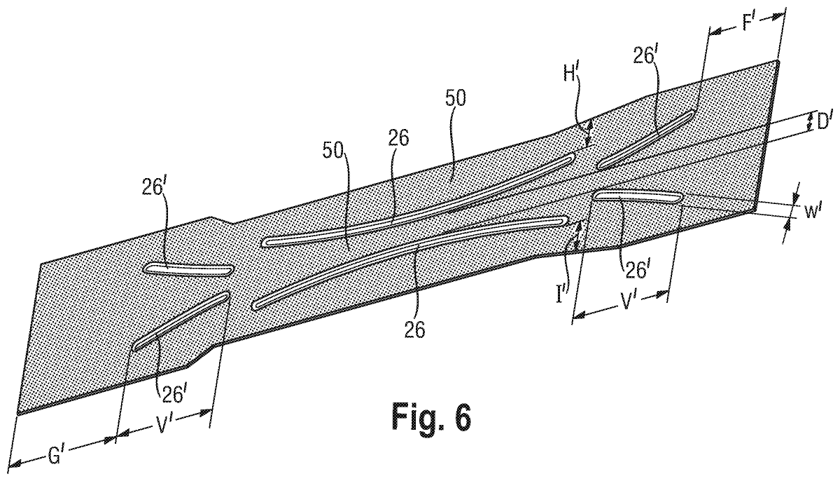

The longitudinal secondary channels may extend over a distance V′ of at least 10%, or at least 15%, or at least 20% of the longitudinal dimension M of the absorbent layer (as shown in FIG. 6). They may extend up to 90% of the longitudinal dimension of the absorbent layer. Typically, the longitudinal secondary channels extend up to 30% or 45% of the longitudinal dimension of the absorbent layer.

Transverse secondary channels may extend over a distance of at least 10%, or at least 15%, or at least 20%, of the transverse dimension (width) N of the absorbent layer. They may extend up to 90% of the transverse dimension of the absorbent layer. Typically, the transverse secondary channels extend up to 30% or 45% of the transverse dimension of the absorbent layer. In some embodiments, the absorbent layer may not comprise transverse channels.

Typically, the secondary channels are distributed such that along each transverse edge of the absorbent layer and immediately adjacent to said edge, the strip which extends in the transverse dimension of the absorbent layer from one longitudinal edge to the other over a distance F′ or G′ remains free of channels (as disclosed above in respect of the longitudinal main channels distribution).

Typically, the secondary channels are distributed such that along each longitudinal edge and immediately adjacent to said edge, the strip which extends in the longitudinal dimension of the absorbent layer from one transverse edge to the other over a distance H′ or I′ remains free of channels (as disclosed above in respect of the longitudinal main channels distribution).

The longitudinal main channels and, when present, the secondary channels may be distributed in the absorbent layer such that a strip extending along the central longitudinal axis of the absorbent layer (and including said axis) from one transverse edge to the other one, and having a width D′ of at least 5%, or at least 10% and up to 60%, or up to 70%, or up to 75% of the transverse dimension of the absorbent layer remains free of channels. The absorbent polymer particles may be continuously present in said strip. For example, said strip may have a width D′ of at least 5 mm, or at least 10 mm, or at least 15 mm or 20 mm and up to 70 mm or up to 40 mm. The absence of channels in said strip is advantageous since it at least inhibits the diaper from taking an inverted V-shape configuration once worn. Inverted V-shape configuration increases the risk of fluid leakages. In some embodiments, the average basis weight of absorbent polymer particles in said strip is high, i.e. at least 350 gsm and up to 1000 gsm, or for example from 450 gsm to 750 gsm.

Although the secondary channels may be transverse secondary channels, the absorbent layer may not comprise any such channels in the crotch region. Channels extending in the transverse dimension in the crotch region would transport liquids to the transverse edges and would increase undesirably the risk of fluid leakages and/or run off. However, such secondary channels may be present in the front region and/or back region of the absorbent layer.

Longitudinal secondary channels may be present in the front region, back region and/or crotch region of the absorbent layer.

As disclosed in respect of the longitudinal main channels, the secondary channels may be straight channels parallel to the longitudinal central axis of the absorbent structure (as shown in FIG. 4A), curved channels (as shown in FIG. 4B), angled channels (as shown in FIG. 4D), oblique channels (as shown in FIG. 4C) or ramified channels (as shown in FIG. 4E). Oblique longitudinal channels, when present in the front or back region of the absorbent layer (i.e. not in the crotch region), may form an angle θ of up to 60 degrees, or up to 50 degrees, or up to 45 degrees with the longitudinal central axis of the absorbent layer.

The secondary channels may have an average width w′ of from 3 mm to 15 mm, or from 4 mm to 14 mm or from 5 mm to 12 mm (the average width of a channel is the average distance between its longitudinal side edges 27) or the average width of the secondary channels may be at least 4% of the width of the absorbent layer, or at least 7% and up to 15%, or 20% or 25%. In some embodiments, the secondary channels may have an average width w′ of from 3 mm to 18 mm, or from 5 mm to 15 mm or from 6 to 10 mm. The ramifications 29, as well, have an average width w′ of at least 3 mm or of at least 4% of the width of the absorbent layer, or at least 7% and up to 15%, or up to 20% or up to 25% (average distance between the longitudinal edges 27′ of the ramifications 29).

The longitudinal main channels and secondary channels may be spaced apart from each other by a distance of at least 5 mm, or at least 8 mm.

The absorbent layer may comprise one or more of said secondary channels, such as 2, 3, 4, 5 or 6. The absorbent layer may comprise an even number of secondary channels. The secondary channels may be distributed in the absorbent layer such that each longitudinal region of the absorbent layer comprises an equal number of secondary channels. In some embodiments, the longitudinal regions comprising the channels (i.e. main longitudinal channels and secondary channels) are minor images of each other with respect to the central longitudinal axis of the absorbent layer.

In some embodiments, such as illustrated in FIGS. 3A to 3C, the longitudinal secondary channels and main longitudinal channels are not distinguishable, i.e. the longitudinal secondary channels and main longitudinal channels are similar. The resulting absorbent layer may thus be seen as comprising more than two longitudinal main channels 26 which are at least present in the crotch region (e.g. 4 longitudinal main channels). In some embodiments, it may be desired that the maximal number of channels in the crotch region is such that the sum of the widths w of the channels 26 is less than 50% of the transverse dimension (width) of the absorbent layer in the crotch region.

In some embodiments, such as shown in FIG. 5, the absorbent layer 17 may comprise two main longitudinal channels 26 as described above and two secondary longitudinal channels in the front region 26′. The two main longitudinal channels 26 are at least present in the crotch region. By at least present in the crotch region, it is meant that said two main longitudinal channels may extend up the front region and/or back region. The two main longitudinal channels 26 at least present in the crotch region may extend over a distance L which is at least 15% of the longitudinal dimension M of the absorbent layer. The secondary longitudinal channels 26′ in the front region may extend over a distance V′ which is at least 10% to 20% of the longitudinal dimension M of the absorbent layer. The two main longitudinal channels 26 in the crotch region may be curved channels whereas the two secondary longitudinal channels 26′ in the front region may be oblique channels. The channels in one of the longitudinal regions may be mirror images of each other in the other longitudinal region.

In some other embodiments, such as shown in FIG. 6, the absorbent layer 17 may comprise two longitudinal main channels 26 in the crotch region, two longitudinal secondary channels 26′ in the front region and two longitudinal secondary channels 26′ in the back region. The longitudinal main channels 26 in the crotch region 25 may extend across at least 15% of the longitudinal dimension of the absorbent layer. The longitudinal secondary channels 26′ in the front region and back region may extend over a distance V′ which is at least 10% to 20% of the longitudinal dimension M of the absorbent layer. The longitudinal main channels 26 in the crotch region may be curved channels whereas the longitudinal secondary channels 26′ in the front region and back region may be oblique channels. The channels in one of the longitudinal regions may be minor images of each other in the other longitudinal region.

When the secondary channels 26′ are longitudinal secondary channels extending in the crotch region, it may be desired that the maximal number of channels in the absorbent layer is such that the sum of the widths w and w′ of the channels (secondary channels and main longitudinal channels) is less than 50% of the transverse dimension of the absorbent layer in the crotch region.

In some embodiment herein, it may be desired that the region 30 of the absorbent layer immediately adjacent the channels 26 and 26′ and extending over a distance k of at least 3 mm, or of at least 5 mm, or of at least 7 mm from the edges of the channels comprises absorbent polymer particles present substantially continuously. In these regions 30, the average basis weight of absorbent polymer particles may be high, i.e. at least 350 gsm, or at least 400 gsm, or at least 500 gsm or at least 600 gsm.

### Absorbent Layer

As explained above, channels 26 and 26′ are regions free of absorbent polymer particles extending through the thickness of the absorbent layer. The absorbent layer comprises absorbent polymer particles 50 alone or in combination with other materials such as cellulose. The absorbent layer may comprise only absorbent polymer particles. The absorbent polymer particles are immobilized on the substrate layer, typically by a thermoplastic adhesive material 40.

Typically the absorbent polymer particles suitable for use in the absorbent layer can comprise any absorbent polymer particles known from superabsorbent literature, for example such as described in Modern Superabsorbent Polymer Technology, F. L. Buchholz, A. T. Graham, Wiley 1998.

The absorbent polymer particles may be spherical, spherical-like or irregular shaped particles, such as Vienna-sausage shaped particles, or ellipsoid shaped particles of the kind typically obtained from inverse phase suspension polymerizations. The particles can also be optionally agglomerated at least to some extent to form larger irregular particles.

The absorbent polymer particles can be selected among polyacrylates and polyacrylate based materials that are internally and/or surface cross-linked, such as for example partially neutralized cross-linked polyacrylates or acid polyacrylate. Examples of absorbent polymer particles suitable in the present disclosure are described for instance in the PCT Patent Application WO 07/047598, WO 07/046052, WO2009/155265 and WO2009/155264.

The absorbent polymer particles may be internally cross-linked, i.e. the polymerization is carried out in the presence of compounds having two or more polymerizable groups which can be free-radically copolymerized into the polymer network. Useful crosslinkers include for example ethylene glycol dimethacrylate, diethylene glycol diacrylate, allyl methacrylate, trimethylolpropane triacrylate, triallylamine, tetraallyloxyethane as described in EP-A 530 438, di- and triacrylates as described in EP-A 547 847, EP-A 559 476, EP-A 632 068, WO 93/21237, WO 03/104299, WO 03/104300, WO 03/104301 and in DE-A 103 31 450, mixed acrylates which, as well as acrylate groups, include further ethylenically unsaturated groups, as described in DE-A 103 31 456 and DE-A 103 55 401, or crosslinker mixtures as described for example in DE-A 195 43 368, DE-A 196 46 484, WO 90/15830 and WO 02/32962 as well as cross-linkers described in WO2009/155265. The absorbent polymer particles may be externally cross-linked (post cross-linked). Useful post-crosslinkers include compounds including two or more groups capable of forming covalent bonds with the carboxylate groups of the polymers. Useful compounds include for example alkoxysilyl compounds, polyaziridines, polyamines, polyamidoamines, di- or polyglycidyl compounds as described in EP-A 083 022, EP-A 543 303 and EP-A 937 736, polyhydric alcohols as described in DE-C 33 14 019, cyclic carbonates as described in DE-A 40 20 780, 2-oxazolidone and its derivatives, such as N-(2-hydroxyethyl)-2-oxazolidone as described in DE-A 198 07 502, bis- and poly-2-oxazolidones as described in DE-A 198 07 992, 2-oxotetrahydro-1,3-oxazine and its derivatives as described in DE-A 198 54 573, N-acyl-2-oxazolidones as described in DE-A 198 54 574, cyclic ureas as described in DE-A 102 04 937, bicyclic amide acetals as described in DE-A 103 34 584, oxetane and cyclic ureas as described in EP-A 1 199 327 and morpholine-2,3-dione and its derivatives as described in WO 03/031482.

The absorbent polymer particles may have surface modifications, such as being coated or partially coated with a coating agent. Examples of coated absorbent polymer particles are disclosed in WO2009/155265. The coating agent may be such that it renders the absorbent polymer particles more hydrophilic. The coating agent may be a polymer, such as an elastic polymer or a film-forming polymer or an elastic film-forming polymer, which forms an elastomeric (elastic) film coating on the particle. The coating may be a homogeneous and/or uniform coating on the surface of the absorbent polymer particles. The coating agent may be applied at a level of from 0.1% to 5%, or from 0.2% to 1% by weight of the surface-modified absorbent polymer particles.

Typically, the absorbent polymer particles may have a selected particle size distribution. For example, the absorbent polymer particles may have a particle size distribution in the range from 45 μm to 4000 μm, more specifically from 45 μm to about 1000 μm, or from about 100 μm to about 850 μm, or from about 100 μm to about 600 μm. The particle size distribution of a material in particulate form can be determined as it is known in the art, for example by means of dry sieve analysis (EDANA 420.02 “Particle Size distribution). Optical methods, e.g. based on light scattering and image analysis techniques, can also be used.

The absorbent layer of the absorbent structure may comprise absorbent polymer particles, and optionally cellulose, distributed on the substrate layer such as to form as a continuous layer, i.e. an uninterrupted layer of absorbent polymeric particles and cellulose when present, which nevertheless comprises regions substantially free of absorbent polymer particles. These discrete regions substantially free of absorbent particles correspond to the channels of the absorbent structure. In some embodiments, the absorbent layer is cellulose free. Alternatively, the absorbent layer may comprise absorbent polymer particles, and optionally cellulose, distributed on the substrate layer such as to form a discontinuous layer. In some embodiments, the absorbent layer is cellulose free. In these embodiments, the absorbent polymer particle and cellulose when present, may be deposited on the substrate layer in clusters of particles (and cellulose when present), thus forming a discontinuous layer or an interrupted layer of absorbent polymer particles (and cellulose when present) which nevertheless comprises regions substantially free of clusters of absorbent polymer particles. These discrete regions substantially free of clusters of absorbent particles correspond to the channels of the absorbent structure. The clusters of absorbent polymer particles (and cellulose when present) may have a variety of shape including, but not limited to, circular, oval, square, rectangular, triangular and the like. Suitable methods for depositing particles in cluster of particles are disclosed in EP 1621167 A2, EP 1913914 A2 and EP 2238953 A2. Typically, absorbent polymer particles are deposited on the substrate layer in clusters of particles when two such absorbent structures are combined to form an absorbent core. The two absorbent structures are combined such that the resulting absorbent core comprises absorbent polymer particles substantially continuously distributed between the two substrate layers, except in regions where channels are present. “Substantially continuously distributed” as used herein indicates that the first substrate layer and second substrate layer are separated by a multiplicity of absorbent polymer particles. It is recognized that there may be minor incidental contact areas between the first substrate layer and second substrate layer within the absorbent particulate polymer material area (i.e. area between the two substrate layers). Incidental contact areas between the first substrate and second substrate may be intentional or unintentional (e.g. manufacturing artifacts) but may not form geometries such as pillows, pockets, tubes, quilted patterns and the like.

The absorbent polymer particles may be immobilized on the substrate layer. Immobilization may be achieved by applying a thermoplastic adhesive material which holds and immobilizes the absorbent polymer particles, and cellulose when present, on the substrate layer. Some thermoplastic adhesive material may also penetrate into the layer of absorbent polymer particles and into the substrate layer to provide further immobilization and affixation. The thermoplastic adhesive material may not only help in immobilizing the absorbent polymer particles on the substrate layer but also helps in maintaining the integrity of the channels. The thermoplastic adhesive material avoids that a significant amount of absorbent polymer particles migrates into the channels.

Integrity/immobilization of the channels may also be achieved by allowing the substrate layer supporting the absorbent polymer particles to fold into the channels, i.e. undulate into the channels. Alternatively, integrity/immobilization may be achieved by allowing a further substrate layer, such as the core cover when present, to fold into the channels, i.e. undulate into the channels. When two absorbent structures as disclosed above are combined, integrity/immobilization may be achieved by allowing the substrate layer of one of the absorbent structures to fold into the channels. In some embodiments, an adhesive (e.g. a thermoplastic adhesive material) may be applied on these portions of the substrate layer which undulates into the channels to provide further affixation.

The thermoplastic adhesive material may be applied as a continuous layer (i.e. uniformly) over the absorbent layer. In some embodiments, the thermoplastic adhesive material contacts the absorbent polymer particles (and cellulose when present) and part of the substrate layer when the absorbent polymer particles (and cellulose when present) are deposited in clusters.

In some embodiments, the thermoplastic adhesive material may be applied as a fibrous layer forming a fibrous network over the absorbent layer. The thermoplastic adhesive fibrous layer may be at least partially in contact with the absorbent polymer particles (and cellulose when present) and partially in contact with the substrate layer of the absorbent structure when the absorbent polymer particles (and cellulose when present) are deposited in clusters. Thereby, the thermoplastic adhesive material may provide cavities to cover the absorbent polymer particles, and thereby immobilizes this material and the channels when present.

Thermoplastic adhesive materials suitable for immobilizing the absorbent polymer particles typically combine good cohesion and good adhesion behavior. Good adhesion promotes good contact between the thermoplastic adhesive material and the absorbent polymer particles and the substrate layer. Good cohesion reduces the likelihood that the adhesive breaks, in particular in response to external forces, and namely in response to strain. When the absorbent structure/core absorbs liquid, the absorbent polymer particles swell and subject the thermoplastic adhesive material to external forces. The thermoplastic adhesive material may allow for such swelling, without breaking and without imparting too many compressive forces, which would restrain the absorbent polymer particles from swelling.

Thermoplastic adhesive materials suitable for use in the present disclosure includes hot melt adhesives comprising at least a thermoplastic polymer in combination with a plasticizer and other thermoplastic diluents such as tackifying resins and additives such as antioxidants. Example suitable hot melt adhesive materials are described in EP 1447067 A2. In some embodiments, the thermoplastic polymer has a molecular weight (Mw) of more than 10,000 and a glass transition temperature (Tg) below room temperature or −6° C.>Tg<16° C. In certain embodiments, the concentrations of the polymer in a hot melt are in the range of about 20 to about 40% by weight. In certain embodiments, thermoplastic polymers may be water insensitive. Example polymers are (styrenic) block copolymers including A-B-A triblock structures, A-B diblock structures and (A-B)n radial block copolymer structures wherein the A blocks are non-elastomeric polymer blocks, typically comprising polystyrene, and the B blocks are unsaturated conjugated diene or (partly) hydrogenated versions of such. The B block is typically isoprene, butadiene, ethylene/butylene (hydrogenated butadiene), ethylene/propylene (hydrogenated isoprene), and mixtures thereof.

Other suitable thermoplastic polymers that may be employed are metallocene polyolefins, which are ethylene polymers prepared using single-site or metallocene catalysts. Therein, at least one comonomer can be polymerized with ethylene to make a copolymer, terpolymer or higher order polymer. Also applicable are amorphous polyolefins or amorphous polyalphaolefins (APAO) which are homopolymers, copolymers or terpolymers of C2 to C8 alpha olefins.

The thermoplastic adhesive material, typically a hot-melt adhesive material, is generally present in the form of fibres, i.e. the hot melt adhesive can be fiberized. In some embodiments, the thermoplastic adhesive material forms a fibrous network over the absorbent polymer particles. Typically, the fibres can have an average thickness from about 1 μm to about 100 μm, or from about 25 μm to about 75 μm, and an average length from about 5 mm to about 50 cm. In particular the layer of hot melt adhesive material can be provided such as to comprise a net-like structure. In certain embodiments the thermoplastic adhesive material is applied at an amount of from 0.5 to 30 g/m², or from 1 to 15 g/m², or from 1 and 10 g/m² or even from 1.5 and 5 g/m² per substrate layer.

A typical parameter for a hot melt adhesive suitable for use in the present disclosure can be a loss angle tan Delta at 60° C. of below the value of 1, or below the value of 0.5. The loss angle tan Delta at 60° C. is correlated with the liquid character of an adhesive at elevated ambient temperatures. The lower tan Delta, the more an adhesive behaves like a solid rather than a liquid, i.e. the lower its tendency to flow or to migrate and the lower the tendency of an adhesive superstructure as described herein to deteriorate or even to collapse over time. This value is hence particularly important if the absorbent article is used in a hot climate.

It may be beneficial, e.g. for process reasons and/or performance reasons, that the thermoplastic adhesive material has a viscosity of between 800 and 4000 mPa·s, or from 1000 mPa·s or 1200 mPa·s or from 1600 mPa·s to 3200 mPa·s or to 3000 mPa·s or to 2800 mPa·s or to 2500 mPa·s, at 175° C., as measurable by ASTM D3236-88, using spindle 27, 20 pmp, 20 minutes preheating at the temperature, and stirring for 10 min.

The thermoplastic adhesive material may have a softening point of between 60° C. and 150° C., or between 75° C. and 135° C., or between 90° C. and 130° C., or between 100° C. and 115° C., as can be determined with ASTM E28-99 (Herzog method; using glycerine).

In one embodiment herein, the thermoplastic adhesive component may be hydrophilic, having a contact angle of less than 90°, or less than 80° or less than 75° or less than 70°, as measurable with ASTM D 5725-99.

In some embodiments, the absorbent structure may also comprise an adhesive material deposited on the substrate before application of the absorbent polymer particles on the substrate layer, referred herein as the auxiliary adhesive. The auxiliary adhesive may enhance the immobilization of the absorbent polymer particles on the substrate layer. It may be a thermoplastic adhesive material, and comprise the same thermoplastic adhesive material as described hereinabove or it may be different. An example of commercially available adhesive is H.B. Fuller Co. (St. Paul, MN) Product No. HL-1620-B. The thermoplastic adhesive material may be applied to the substrate layer by any suitable means.

In some embodiments, the absorbent polymer particles, and optionally cellulose, may be distributed evenly in the longitudinal and/or transverse dimensions of the absorbent layers irrespective of whether the absorbent layer is a continuous layer or a discontinuous layer of absorbent polymer particles, and optionally cellulose, as described above to provide an absorbent core having an even distribution of absorbent polymer particles. The average basis weight of absorbent polymer particles may depend on the particular diaper in which it may be incorporated. In some embodiments, the average basis weight of absorbent polymer particles in the absorbent core may be from 350 gsm to 1500 gsm. The amount of absorbent polymeric particles in the respective region is calculated by determining the weight of absorbent polymeric material in this region and dividing it by the total surface area of the region (hence, the average amount is taken).

In some embodiments, the absorbent polymer particles, and optionally cellulose, may be distributed unevenly in the longitudinal and/or transverse dimension of at least one of the absorbent layers to provide a profiled absorbent core. For example, the crotch region of the absorbent structure/core may comprise a higher amount of absorbent polymer particles per area compared to the front and back regions of the absorbent structure/core. In some embodiment, the front half of the absorbent core comprises most of the absorbent capacity, i.e. it may comprise more than about 60% of the absorbent polymer particles, or more than about 65%, or more than 70%, based on the total weight of absorbent polymer particles in the absorbent core.

In some embodiments, the absorbent structure as disclosed above may form the absorbent core of the disposable diaper or may be a component thereof (i.e. the absorbent core may be a laminate of absorbent structures).

The absorbent core may further comprise a cover layer, typically disposed on the thermoplastic adhesive material. The cover layer may be a separate layer or it may be unitary with the substrate layer. In such a case, the substrate layer supporting the absorbent polymer particles is folded to form a top and bottom layer which encloses the absorbent polymer particles. The cover layer may be provided of the same material as the substrate layer, or may be provided of a different material. The layers may be bonded together at about the periphery to enclose the absorbent polymer particles therein, e.g. by adhesive bonding and/or heat bonding. In some embodiments, the core cover may undulate into the channels.

In some embodiments, the absorbent core may comprise an acquisition system which is disposed between the topsheet and the wearer facing side of the absorbent structure. The acquisition system may serve as a temporary reservoir for liquid until the absorbent structure can absorb the liquid. The acquisition system may comprise a single layer or multiple layers, such as an upper acquisition layer facing towards the wearer's skin and a lower acquisition layer facing the garment of the wearer. The acquisition system may be in direct contact with the absorbent structure. In these embodiments, the acquisition system may fill in the channels or part thereof. In some embodiments, the acquisition system may be placed on top of the core cover when present. In embodiments wherein the core cover or substrate layer folds into the channels, i.e. undulates into the channels, the acquisition system may fill in the channels or part thereof. In some embodiments, the acquisition system, or one layer thereof, may be bonded to the core cover or substrate layer which undulates into the channels thus providing an undulating profile to said acquisition system.

In a certain embodiment, the acquisition system may comprise chemically cross-linked cellulosic fibers. Such cross-linked cellulosic fibers may have desirable absorbency properties. Example chemically cross-linked cellulosic fibers are disclosed in U.S. Pat. No. 5,137,537. In certain embodiments, the chemically cross-linked cellulosic fibers are cross-linked with between about 0.5 mole % and about 10.0 mole % of a C₂ to C₉ polycarboxylic cross-linking agent or between about 1.5 mole % and about 6.0 mole % of a C₂ to C₉ polycarboxylic cross-linking agent based on glucose unit. Citric acid is an example cross-linking agent. In other embodiments, polyacrylic acids may be used. Further, according to certain embodiments, the cross-linked cellulosic fibers have a water retention value of about 25 to about 60, or about 28 to about 50, or about 30 to about 45. A method for determining water retention value is disclosed in U.S. Pat. No. 5,137,537. According to certain embodiments, the cross-linked cellulosic fibers may be crimped, twisted, or curled, or a combination thereof including crimped, twisted, and curled.

In a certain embodiment, one or both of the upper and lower acquisition layers may comprise a non-woven, which may be hydrophilic. Further, according to a certain embodiment, one or both of the upper and lower acquisition layers may comprise the chemically cross-linked cellulosic fibers, which may or may not form part of a nonwoven material. According to an example embodiment, the upper acquisition layer may comprise a nonwoven, without the cross-linked cellulosic fibers, and the lower acquisition layer may comprise the chemically cross-linked cellulosic fibers. Further, according to an embodiment, the lower acquisition layer may comprise the chemically cross-linked cellulosic fibers mixed with other fibers such as natural or synthetic polymeric fibers. According to example embodiments, such other natural or synthetic polymeric fibers may include high surface area fibers, thermoplastic binding fibers, polyethylene fibers, polypropylene fibers, PET fibers, rayon fibers, lyocell fibers, and mixtures thereof. Suitable non-woven materials for the upper and lower acquisition layers include, but are not limited to SMS material, comprising a spunbonded, a melt-blown and a further spunbonded layer. In certain embodiments, permanently hydrophilic non-wovens, and in particular, nonwovens with durably hydrophilic coatings are desirable. Another suitable embodiment comprises a SMMS-structure. In certain embodiments, the non-wovens are porous.

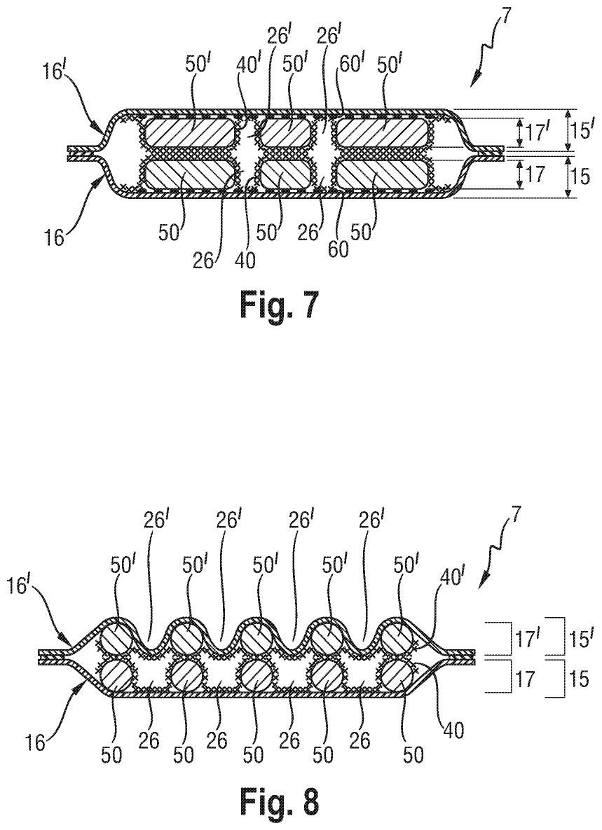

In some embodiments, as shown in FIGS. 7 to 10, the absorbent core of the disposable diaper may comprise two or more absorbent structures as disclosed herein, i.e. absorbent structures which comprise channels, which are combined or superposed. Typically, the absorbent structures may be combined such that the thermoplastic adhesive material of the first absorbent structure directly contacts the thermoplastic adhesive material of the second absorbent structure.

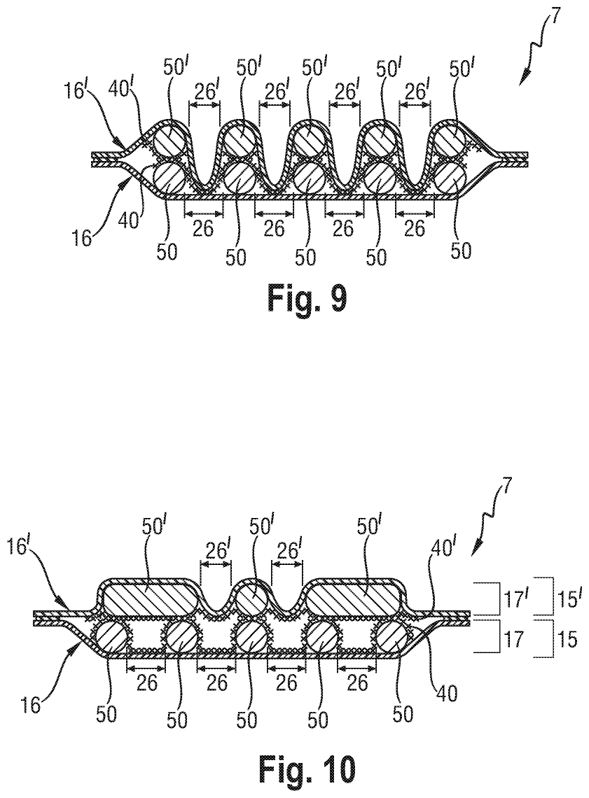

FIGS. 7, 8, 9 and 10 illustrate embodiments wherein a first absorbent structure 15 comprising an absorbent layer 17 with channels 26 is combined with a second absorbent structure 15′ comprising an absorbent layer 17′ with channels 26′. In the shown embodiments, the thermoplastic adhesive material 40 of the first absorbent structure directly contacts the thermoplastic adhesive material 40′ of the second absorbent structure. An auxiliary adhesive 60 may be present on the substrate layer of the first and/or second absorbent structure for further immobilization of the absorbent polymer particles (as shown in FIG. 7). In some embodiments, the substrate layer 16′ of the second absorbent structure 15′ may undulate into the channels 26′ of the second absorbent structure (as shown in FIGS. 8 and 10) and even into the channels 26 of the first absorbent structure 15, where it can be possibly adhered to the substrate layer 16 of the first absorbent structure (as shown in FIG. 9). Typically, when the substrate layer of one absorbent structure undulates into the channels, the substrate layers of the two absorbent structures are not coextensive, i.e. one of the substrate layer may be wider or longer to penetrate into the channels 26 and/or 26′. Undulations of the substrate layer into the channels contribute to the integrity of the channels in dry and wet states.

In embodiments wherein the two or more absorbent structures comprise channels, it may be that the first and second absorbent structures are mirror images of each other. In these embodiments, the channels 26 of the first absorbent structure 15 substantially superpose the channels 26′ of the adjacent second absorbent structure 26′, as shown in FIGS. 7, 8 and 9. The resulting absorbent core is a laminate of absorbent structures 17 and 17′ with channels extending substantially through the thickness of the absorbent core (by substantially as used herein it is meant that the thicknesses of the substrate layers are hereby neglected).

In embodiments wherein the two or more absorbent structures comprise channels, the first and second absorbent structures may be different. In some of these embodiments, some of the channels of the two absorbent layers may superpose (as shown in FIG. 10). In other embodiments, the channels of one absorbent structure may not superpose the channels of the adjacent absorbent structure but are complementary with the channels of the adjacent structure. By complementary it is meant that the channels of the second absorbent structure form an extension of the channels of the first absorbent structure.

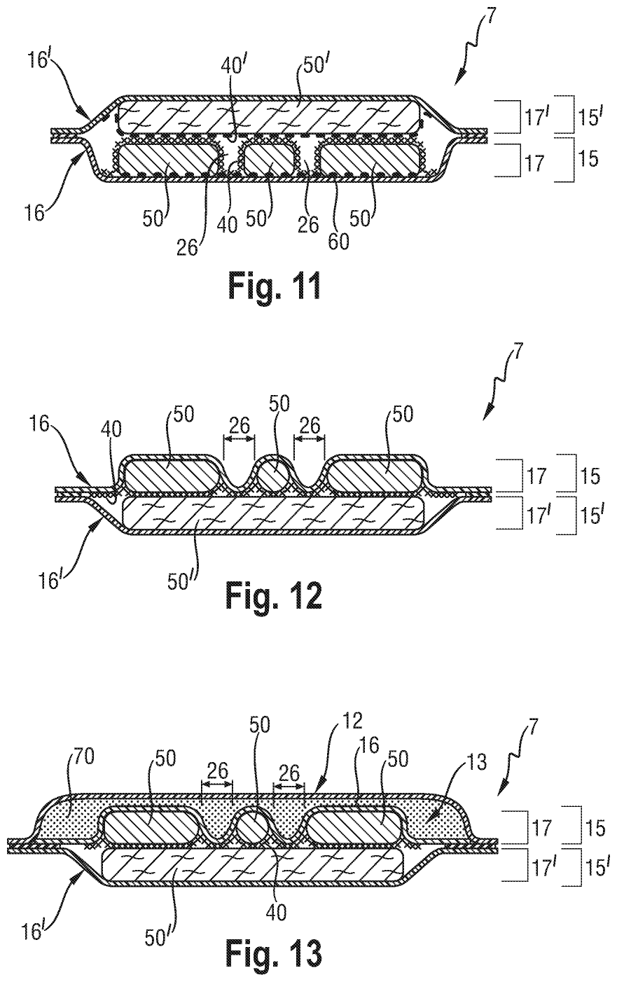

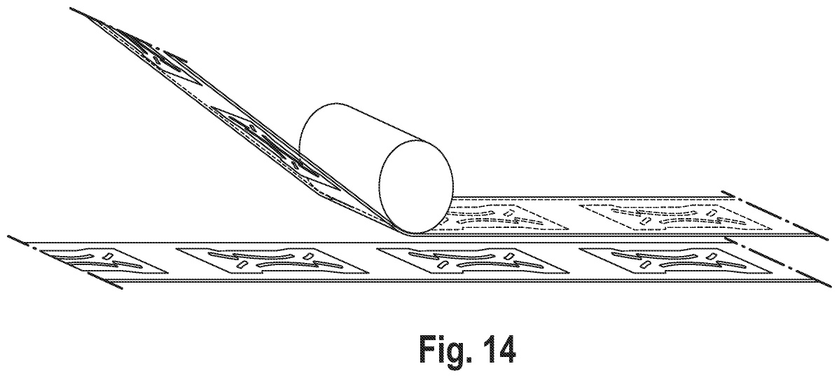

In some embodiments, as shown in FIGS. 11 to 13, the absorbent core of the disposable diaper may comprise one or more absorbent structures as disclosed herein, combined with an absorbent structure comprising an absorbent layer which is free of channels. In these embodiments, the absorbent structure free of channels comprises a substrate layer and an absorbent layer as disclosed which is however free of channels. When only one of the absorbent structures comprises said channels, the absorbent structure with channels may in some embodiments herein be closer to the wearer in use than the absorbent structure(s) without channels.

FIGS. 11, 12 and 13 illustrate embodiments wherein a first absorbent structure 15 with channels 26 is combined with a second absorbent structure 15′ free of channels. In the embodiment shown in FIG. 11, the thermoplastic adhesive material 40 of the first absorbent structure 15 directly contacts the thermoplastic adhesive material 40′ of the second absorbent structure 15′ whereas in FIGS. 12 and 13, the second absorbent structure 15′ may not comprise any such thermoplastic adhesive material 40′ (however, it may be present to immobilize the absorbent layer on the substrate layer). An auxiliary adhesive 60 may be present on the substrate layer of the first and/or second absorbent structure for further immobilization of the absorbent polymer particles 50 and 50′ (as shown in FIGS. 11 and 13). In some embodiments, the substrate layer 16 of the first absorbent structure 15 may undulate into the channels 26 of the first absorbent structure (as shown in FIGS. 12 and 13). The absorbent core may further comprise an acquisition system 70 that penetrates and fill in the channels (however in some embodiments, the acquisition system may not fill in the channels). FIG. 13 shows an embodiment wherein the acquisition system 70 comprises a first layer 12 and second layer 13, wherein the second layer fills in the channels.

### Method of Making the Absorbent Structure

The absorbent structure having channels herein may be made by any method comprising the step of depositing absorbent polymer particles and optionally cellulose to form an absorbent layer onto a substrate layer, for example by placing first said substrate layer onto raised portions in the shape and dimensions of said channels to be produced and then depositing said absorbent polymer particles and optionally cellulose thereon; thereby, the absorbent polymer particles and optionally cellulose may not remain onto said raised portions, but only on the remaining portions of the substrate layer.

The absorbent structure free of channels herein may be made by any method comprising the step of depositing absorbent polymer particles and optionally cellulose as an absorbent layer on a substrate layer.

In some embodiments, the absorbent structure with the substrate layer with therein two or more channels with substantially no absorbent material is for example obtainable by a method comprising the steps of:
a) providing a feeder for feeding said absorbent material (absorbent polymer particles and optionally cellulose) to a first moving endless surface, such as a hopper;
b) providing a transfer means for transferring a substrate layer to a second moving endless surface;
c) providing a first moving endless surface, having one or more absorbent layer-forming reservoirs with a longitudinal dimension and averaged length, a perpendicular transverse dimension and average width, and, perpendicular to both, a depth dimension and average depth, and a void volume for receiving said absorbent material therein, said reservoir(s) comprising one or more substantially longitudinally extending raised strips, not having a void volume, for example each having an average width W of at least 4% or at least 5% of the average width of the reservoir, and an average length L of at least 5% and at the most 30% of the average longitudinal dimension of the reservoir; said reservoir(s) being for transferring said absorbent material to said second moving endless surface adjacent and in proximity thereto;
d) providing a second moving surface, having an outer shell that has one or more air permeable or partially air permeable receptacles with for receiving said substrate layer thereon or therein, with a receiving area and with one or more substantially longitudinally extending mating strips that may be air impermeable, and having each an average width of for example W′ of at least 2.5 mm, optionally from 0.5×W to 1.2×W, an average length of for example L′ being from about 0.8×L to 1.2×L;
whereby said air-permeable outer shell is connected to one or more secondary vacuum systems for facilitating retention of the substrate layer and/or said absorbent material thereon, and
whereby, in a meeting point, said first moving endless surface and said outer shell are at least partially adjacent to one another and in close proximity of one another during transfer of said absorbent material and such that each mating strip is substantially completely adjacent and in close proximity to a raised strip during transfer of said absorbent material;
e) feeding with said feeder an absorbent material to said first moving endless surface, in at least said reservoir (s) thereof;
f) optionally, removing any absorbent material on said raised strips (s);
g) simultaneously, transferring said substrate layer to said second moving endless surface, onto or into said receptacle(s);
h) selectively transferring in said meeting point, said absorbent material with said first moving endless surface only to said part of the supporting sheet that is on or in said receiving area of said receptacle.

Said reservoir(s) may be formed by of a multitude of grooves and/or cavities with a void volume, for receiving said absorbent material therein. In some embodiments, the average width W of (each) strip may be at least 6 mm, or for example at least 7 mm, and/or at least at least 7%, or for example at least 10% of the average width of the respective reservoir.

Said grooves and/or cavities may each for example have a maximum dimension in transverse direction which is at least 3 mm, and whereby the shortest distance between directly neighboring cavities and/or grooves in substantially transverse dimension, is less than 5 mm. Cavities and/or grooves that are directly adjacent a raised strip may have a volume that is more than the volume of one or more, or all of their neighboring cavities or grooves, that are not directly adjacent said strip or another strip (thus further removed from a strip).

Said first moving endless surface's reservoir may be at least partially air permeable and said first moving endless surface may have a cylindrical surface with said reservoirs, rotatably moving around a stator, comprising a vacuum chamber; said second moving surface's outer shell may be cylindrical, rotatably moving around a stator, comprising a secondary vacuum chamber connected to said secondary vacuum system.

The method further comprises the step of i) applying an adhesive material to the absorbent structure of step h; and optionally applying an adhesive material (i.e. a second adhesive material) to said substrate layer, prior to step f, or simultaneously therewith, but in any event prior to step g).

Step i) 1) may involve straying said first adhesive material in the form of fibers onto said absorbent layer, or part thereof, for example substantially continuously, so it is also present in said channels.

Step i) 2) may involve slot coating or spray-coating the supporting sheet, either continuously, or for example in a pattern corresponding to the channel pattern. An absorbent structure obtained by said method can then be combined with an absorbent structure free of channels or with another absorbent structure made by this method to provide an absorbent core.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for making an absorbent structure for use in a disposable absorbent article, the method comprising the steps of:
providing a first moving surface comprising an absorbent layer-forming reservoir having one or more substantially longitudinally extending raised strips, wherein the one or more substantially longitudinally extending raised strips have an average width of between 5% and 30% of an average width of the absorbent layer-forming reservoir, and wherein the first moving surface is partially air permeable;
providing a supply of absorbent material comprising superabsorbent polymer particulate material;
depositing a portion of the supply of absorbent material into the absorbent layer-forming reservoir;
associating the portion of the supply of absorbent material with a first substrate layer and/or a second substrate layer to define an absorbent structure comprising a channel therein corresponding to the one or more raised strips; and
bonding the first substrate layer to the second substrate layer through the channel,
wherein the channel is substantially free of the supply of absorbent material.

2. The method of claim 1, wherein the first substrate layer is bonded to the second substrate layer via glue.

3. The method of claim 1, wherein the first substrate layer is bonded to the second substrate layer via ultrasonic bonding.

4. The method of claim 1, wherein the first substrate layer is bonded to the second substrate layer via heat bonding.

5. The method of claim 1, further comprising the step of applying a first adhesive material to the first substrate layer and/or the second substrate layer prior to associating the portion of the supply of absorbent material with the first substrate layer and/or the second substrate layer.

6. The method of claim 1, wherein the first substrate layer is continuously bonded to the second substrate layer in the channel.

7. The method of claim 6, wherein the bonding of the first substrate layer to the second substrate layer in the channel is permanent.

8. The method of claim 1, wherein the first substrate layer is intermittently bonded to the second substrate layer in the channel.

9. The method of claim 1, wherein the one or more substantially longitudinally extending raised strips have an average length of 5% to 30% of an average longitudinal dimension of the absorbent layer-forming reservoir.

10. The method of claim 1, further comprising the step of applying a second adhesive material onto the portion of the supply of absorbent material after associating the portion of the supply of absorbent material with the first substrate layer and/or the second substrate layer.

11. The method of claim 10, wherein the second adhesive material is in the form of fibers.

12. The method of claim 1, further comprising the step of removing absorbent material from the one or more substantially longitudinally extending raised strips after depositing the portion of the supply of absorbent material into the absorbent layer-forming reservoir.

13. The method of claim 1, further comprising the step of providing a second moving surface, wherein the second moving surface comprises an air permeable receptacle for receiving the first substrate layer or the second substrate layer.

14. The method of claim 13, wherein the second moving surface comprises one or more longitudinally extending mating strips, wherein the one or more longitudinally extending mating strips are air impermeable.

15. The method of claim 14, wherein upon associating the portion of the supply of absorbent material with the first substrate layer and/or the second substrate layer, the one or more substantially longitudinally extending raised strips are substantially completely adjacent and in close proximity to the one or more longitudinally extending mating strips.

16. The method of claim 1, wherein the supply of absorbent material comprises cellulosic material.
